(19) United States
(12) Patent Application Publication
(10) Pub. No.: US 2013/0053844 A1
(43) Pub. Date: Feb. 28, 2013

(54) TREATMENT INSTRUMENT FOR ENDOSCOPE

(71) Applicants: Keita SUZUKI, Tokyo (JP); Megumi KIMURA, Tokyo (JP)

(72) Inventors: Keita SUZUKI, Tokyo (JP); Megumi KIMURA, Tokyo (JP)

(73) Assignee: OLYMPUS MEDICAL SYSTEMS CORP., Tokyo (JP)

(21) Appl. No.: 13/591,909

(22) Filed: Aug. 22, 2012

Related U.S. Application Data
(63) Continuation of application No. PCT/JP2012/054777, filed on Feb. 27, 2012.

(30) Foreign Application Priority Data
Mar. 2, 2011 (JP) 2011-045031

(51) Int. Cl. A61B 18/14

(52) U.S. Cl. 606/46

(57) ABSTRACT

This treatment instrument for endoscope includes a sheath that is capable of being inserted into a treatment instrument channel of an endoscope, a treatment part provided on a distal-end side of the sheath, a first wire that is connected to the treatment part and inserted into the sheath, the first wire including a first core member and a first covering member that covers the first core member, and a second wire that drives the treatment part, connected to the treatment part and inserted into the sheath so that the second wire is capable of moving relatively with the first wire in a state where an outer peripheral surface of the second wire capable of contacting the first wire, the second wire including a second core member and a second covering member having a different surface condition from that of the first covering member.

The present application is a continuing application based on PCT Patent Application No. PCT/JP2012/054777, filed on Feb. 27, 2012, whose priority is claimed on Japanese Patent Application No. 2011-045031, filed on Mar. 2, 2011. The contents of both the PCT Application and the Japanese Patent Application are incorporated herein by reference.

### BACKGROUND OF THE INVENTION

1. Field of the Invention

The present invention relates to a treatment instrument for endoscope.

2. Description of Related Art

Conventionally, a treatment instrument for endoscope is known that is used to perform surgery using an endoscope. As an example of this type of treatment instrument for endoscope, for example, Japanese Unexamined Patent Application, First Publication No. 2004-187 describes a treatment instrument wherein two operation wires (operation shaft members), the tips whereof are respectively connected to a pair of forceps members, can move relatively and are provided inside a sheath member formed into a cylindrical shape. With the treatment instrument described in Japanese Unexamined Patent Application, First Publication No. 2004-187, the pair of forceps members can be operated by moving two operation wires forward and backward inside the sheath member.

Furthermore, Japanese Unexamined Patent Application, First Publication No. 2010-17224 describes a treatment instrument (high-frequency treatment instrument) wherein an operation wire for operating a pair of forceps members, and a power-supply wire for passing a high-frequency current through the pair of forceps members are each provided inside a sheath member (coil sheath). With the treatment instrument described in Japanese Unexamined Patent Application, First Publication No. 2010-17224, the pair of forceps members can be opened and closed by moving the operation wire forward and backward inside the sheath member.

### SUMMARY OF THE INVENTION

According to a first aspect of the present invention, a treatment instrument for endoscope includes a sheath, a treatment part, a first wire, and a second wire. The sheath is capable of being inserted into a treatment instrument channel of an endoscope. The treatment part is provided on a distal-end side of the sheath. The first wire is connected to the treatment part and inserted into the sheath. The first wire includes a first core member and a first covering member that covers the first core member. To drive the treatment part, the second wire is connected to the treatment part and inserted into the sheath so that it is capable of moving relatively with the first wire in a state where an outer peripheral surface of the second wire is capable of contacting the first wire. The second wire includes a second core member, and a second covering member having a different surface condition from that of the first covering member.

According to a second aspect of the present invention, the first wire is a power-supply wire that supplies electrical power to the treatment part.

According to a third aspect of the present invention, outer peripheral surfaces of the first covering member and the second covering member have mutually different coefficients of friction.

According to a fourth aspect of the present invention, the first covering member and the second covering member are made from different materials.

According to a fifth aspect of the invention, a first concavoconvex pattern is formed in the outer peripheral surface of the first covering member.

According to a sixth aspect of the invention, a second concavoconvex pattern having a different shape to that of the first concavoconvex pattern is formed in the outer peripheral surface of the second covering member, by a formation method that is different from that of the first concavoconvex pattern.

According to a seventh aspect of the present invention, the first covering member is made from a material having an electrical insulating effect.

According to an eighth aspect of the present invention, the outer peripheral surfaces of the first covering member and the second covering member have mutually different microscopic structures.

### BRIEF DESCRIPTION OF THE DRAWINGS

FIG. 1 is a side view showing a treatment instrument for endoscope in one embodiment of the present invention.

FIG. 2 is a partial cross-sectional view showing the configuration of a treatment part vicinity of a treatment instrument for endoscope in one embodiment of the present invention.

FIG. 3 is an explanatory view of an operation of a treatment part of a treatment instrument for endoscope in one embodiment of the present invention.

FIG. 4 is a partial cross-sectional view of a treatment part of a treatment instrument for endoscope in one embodiment of the present invention, and shows a view in a direction of an arrow A in FIG. 2.

FIG. 5 is a partial cross-sectional view of a treatment part of a treatment instrument for endoscope in one embodiment of the present invention, and shows a view in a direction of an arrow B in FIG. 4.

FIG. 6 is a cross-sectional view taken along the line C-C in FIG. 4.

FIG. 7 is a view showing the configuration of a modified example (first modified example) of one embodiment of the present invention, and shows a cross-sectional view taken along the line D-D in FIG. 6.

FIG. 8 is an enlarged view showing the section in FIG. 7 indicated by symbol X.

FIG. 9 is a view of another example configuration of a modified example (second modified example) of one embodiment of the present invention, and shows an enlarged view of a section corresponding to the section in FIG. 7 indicated by symbol X.

FIG. 10 is a view showing another example configuration (fourth modified example) of a treatment instrument for endoscope in one embodiment of the present invention, and shows an enlarged view of a section corresponding to the section in FIG. 7 indicated by symbol X.

FIG. 11 is a view showing yet another example configuration of a modified example (fifth modified example) of a treatment instrument for endoscope in one embodiment of the present invention, and shows an enlarged view of a section corresponding to the section in FIG. 7 indicated by symbol X.

### DETAILED DESCRIPTION OF THE INVENTION

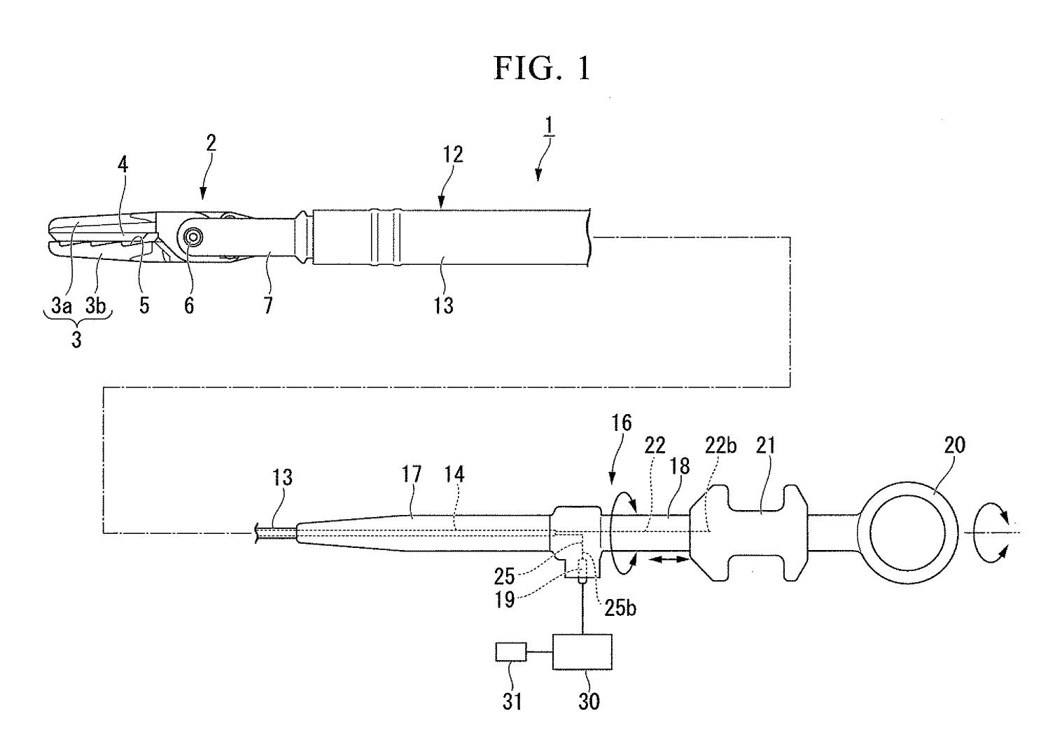

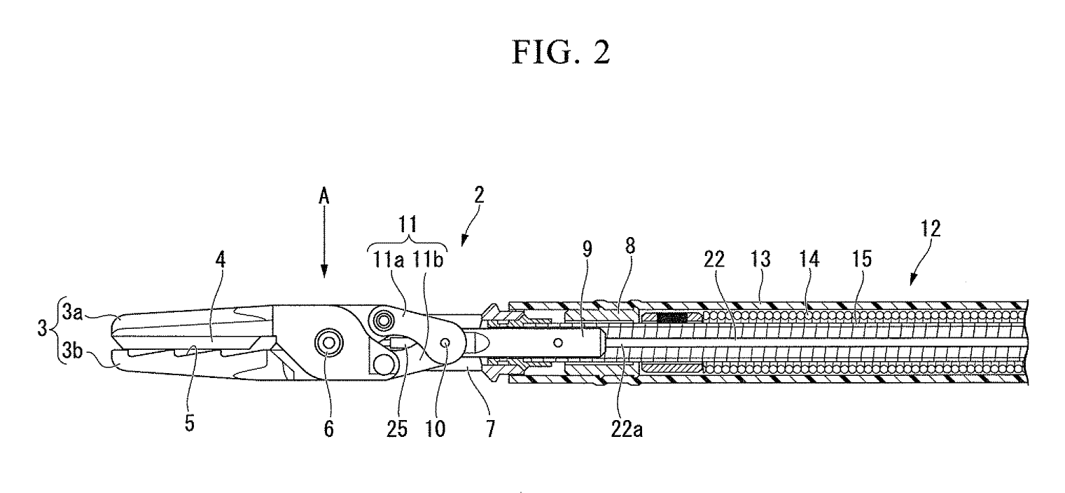

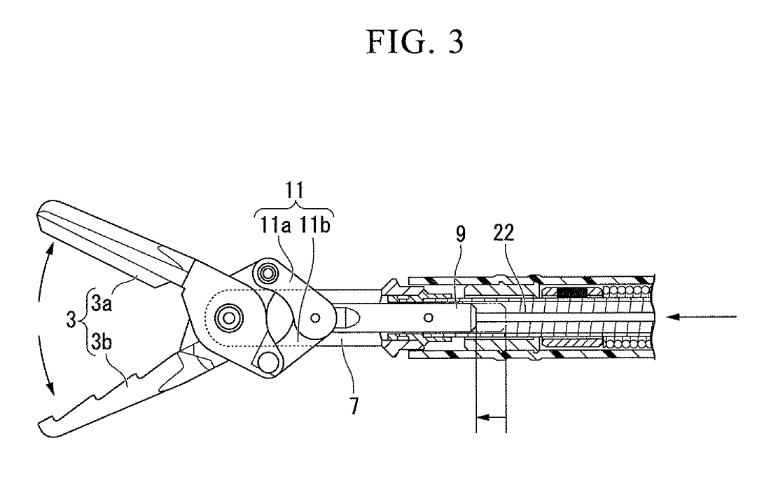

A treatment instrument for endoscope in one embodiment of the invention will be explained. FIG. 1 is a side view showing a treatment instrument for endoscope 1 in this embodiment. FIG. 2 is a partial cross-sectional view showing the configuration of a treatment part 2 vicinity of the treatment instrument for endoscope 1. FIG. 3 is an explanatory view of an operation of the treatment part 2 of the treatment instrument for endoscope 1.

The treatment instrument for endoscope 1 of the embodiment is a treatment instrument for carrying out medical treatment to a living tissue. The treatment instrument for endoscope 1 is inserted into a treatment instrument channel of an endoscope, and used with the endoscope.

As shown in FIGS. 1 and 2, the treatment instrument for endoscope 1 includes a treatment part 2 for carrying out treatment to a living tissue, an elongated and flexible insertion part 12, the treatment part 2 being attached to the distal end of the insertion part 12, an operation part 16 provided at the proximal end of the insertion part 12, an operation wire 22 (first wire), and a power-supply wire 25 (second wire). The power-supply wire 25 is provided inside the insertion part 12.

As shown in FIG. 2, the treatment part 2 includes a pair of forceps members 3 (first forceps member 3a and second forceps member 3b) for gripping the living tissue, a transmitting member 9 fixed to the distal end of the operation wire 22, a pair of linking members 11 (first linking member 1.1a and second linking member 11b) for connecting the pair of forceps members 3 to the transmitting member 9, and a covering member 7 that supports the connecting rod 3 such that they can open and close.

The first forceps member 3a has a wire-shaped incision electrode 4 for passing a high-frequency current through the living tissue. The incision electrode 4 is electrically connected to the power-supply wire 25. The incision electrode 4 is configured such that a high-frequency current is supplied via the power-supply wire 25. The outer surface of the first forceps member 3a is coated with an insulating material, excepting the two points where it connects to the incision electrode 4 and to the connection point between the first forceps member 3a and the power-supply wire 25.

In the treatment instrument for endoscope 1 of this embodiment, the high-frequency current supplied to the incision electrode 4 flows through the body of the patient on whom the treatment instrument for endoscope 1 is being used to a return electrode 31 described below (see FIG. 1). That is, the treatment instrument for endoscope 1 of the embodiment is a so-called monopolar high-frequency treatment instrument.

The second forceps member 3b has a forceps face 5 with a saw-toothed shape that faces the incision electrode 4 of the first forceps member 3a.

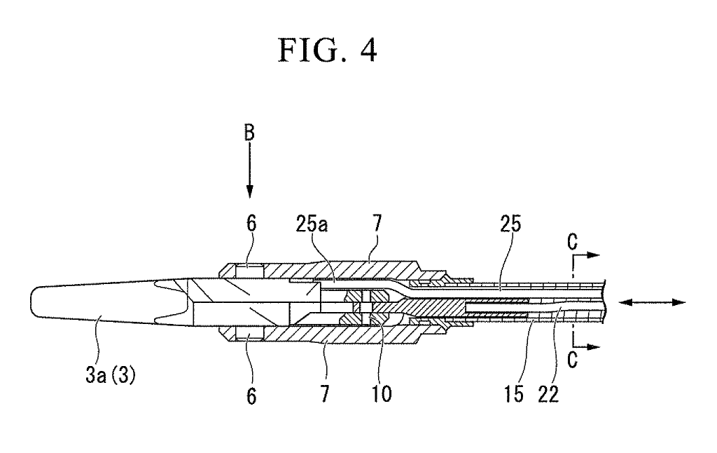

The first forceps member 3a and the second forceps member 3b are mutually rotatably connected at a connecting shaft part 6. Both ends of a connecting member are connected to the covering member 7 (see FIG. 4).

FIG. 4 is a partial cross-sectional view of the treatment instrument for endoscope 1, and shows a view in a direction of an arrow A of FIG. 2.

As shown in FIGS. 2 and 4, the covering member 7 is fixed to an inner coil sheath 15 described below. The covering member 7 is configured to be rotatable about the center axis of rotation of an outer sheath 13 constituting the outermost layer of the insertion part 12, Thus, when the inner coil sheath 15 is rotated around the center axis of rotation within the insertion part 12, the covering member 7 and the pair of forceps members 3 rotate in conjunction with the rotation of the inner coil sheath 15.

As shown in FIG. 2, a substantially cylindrical supporting member 8 is provided between the covering member 7 and the outer sheath 13. The supporting member 8 is fixed to the inner peripheral surface of the outer sheath 13, and the covering member 7 is rotatably inserted into the supporting member 8.

The transmitting member 9 is provided inside the covering member 7. One part of the proximal end of the transmitting member 9 is configured to be capable of being inserted inside the inner coil sheath 15 described below. The distal end of the transmitting member 9 is rotatably connected to the first linking member 1 la and the second linking member 11b by a pin 10.

The first linking member 11a and the second linking member 11b are rotatably connected to the transmitting member 9 by the pin 10, and are rotatably connected to each of the first forceps member 3a and the second forceps member 3b. The first linking member 11a and the second linking member 11b convert the amount of actuating force (pressing force and traction force) that moves the operation wire 22 forward and backward in the direction of the center axis of the insertion part 12 to a driving force that opens and closes the pair of forceps members 3.

The insertion part 12 includes an outer sheath 13 formed from a tube-shaped resin, an outer coil sheath 14 inserted inside the outer sheath 13, and an inner coil sheath 15 inserted inside the outer coil sheath 14.

The outer sheath 13 is a member provided to the insertion part 12 with the aim of preventing liquid and the like from infiltrating inside the insertion part 12, and is flexible.

The outer coil sheath 14 is a sheath formed by winding a metal wire that is circular in cross-section into a coil shape, and is flexible.

The inner coil sheath 15 is a flat coil sheath formed by winding a metal wire that is rectangular in cross-section into a coil shape, and is flexible.

The distal end of the inner coil sheath 15 is fixed to the distal end of the outer coil sheath 14 by a method such as, for example, soldering or laser welding. Thus the inner coil sheath 15 and the outer coil sheath 14 integrally rotate with respect to the outer sheath 13.

In the embodiment, the outer coil sheath 14 and the inner coil sheath 15 are arranged coaxially within the outer sheath 13. It is thus possible to obtain both a resistance to compression in the direction of the center axis of the insertion part 12 and a rotational following performance in the center axis direction of the insertion part 12. Furthermore, the insertion part 12 is entirely flexible, and can be inserted into a treatment instrument channel of a flexible endoscope.

As shown in FIG. 1, the operation part 16 includes a cylindrical main-body part 17, a rod-shaped rotation-operation body 18, and a slider 21, The proximal ends of the outer coil sheath 14 and the inner coil sheath 15 are fixed to the rotation-operation body 18. The slider 21 is connected so that it can move forward and backward in the direction of the longitudinal axis of the rotation-operation body 18.

The proximal end of the outer sheath 13 is fixed to the main-body part 17. In addition, the outer coil sheath 14 and the inner coil sheath 15 are rotatably inserted inside the main-body part 17.

The rotation-operation body 18 is rotatably connected to the main-body part 17. The rotation-operation body 18 can rotate the outer coil sheath 14, the inner coil sheath 15, the operation wire 22, and the power-supply wire 25 shown in FIG. 2 integrally with respect to the main-body part 17. As shown in FIG. 1, the rotation-operation body 18 is provided with a terminal part 19 which can be connected to a high-frequency power source device 30 (power-supply device). The high-frequency power source device 30 supplies high-frequency current (electrical energy) to the power-supply wire 25. The proximal end 25b of the power-supply wire 25 is fixed to the terminal part 19. A ring-shaped finger-hook part 20 for enabling a user of the treatment instrument for endoscope 1 to hook his fingers is formed at the proximal end of the rotation-operation body 18.

In the embodiment, the high-frequency power source device 30 includes a return electrode 31 used by attaching it to the body-surface of the patient on whom the treatment instrument for endoscope 1 is to be used. The high-frequency current supplied to the incision electrode 4 of the treatment instrument for endoscope 1 thus flows through the body of the patient to the return electrode 31.

The outer surface of the slider 21 is formed with a concave shape so that the user of the treatment instrument for endoscope 1 can hook his fingers around it. The proximal end 22b of the operation wire 22 is fixed to the slider 21. The slider 21 moves in the direction of the longitudinal axis of the rotation-operation body 18 by an operation of the user. As a result, the force that the user applies to the slider 21 is transmitted to the operation wire 22, and the operation wire 22 moves forward and backward in the direction of its center axis.

In the operation part 16, when the slider 21 moves forward and backward with respect to the rotation-operation body 18, a driving force for opening and closing the pair of forceps members 3 is transmitted through the operation wire 22 to the treatment part 2. In the embodiment, the force that the user applies to the slider 21 when he moves it forward and backward is the amount of actuating force in the treatment instrument for endoscope 1.

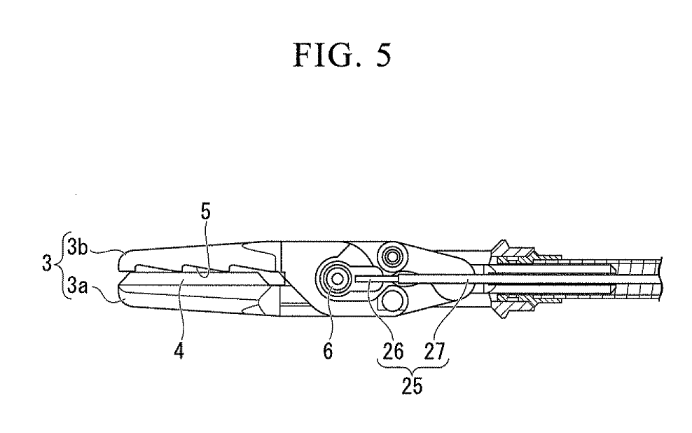

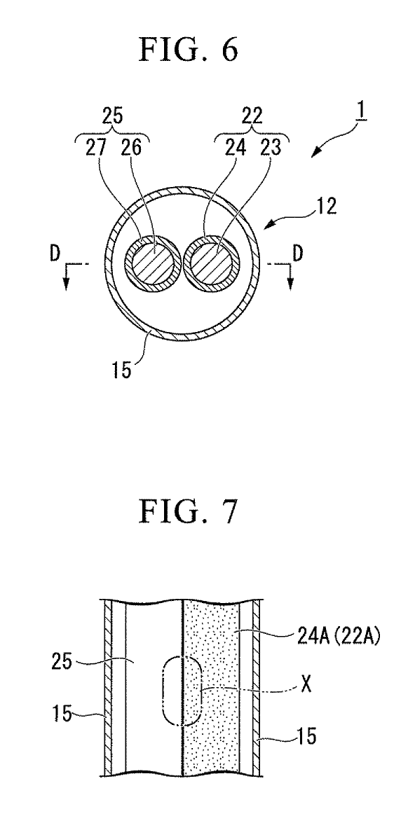

FIG. 5 is a partial cross-sectional view of the treatment instrument for endoscope 1, and shows a view in a direction of an arrow B in FIG. 4, FIG. 6 is a cross-sectional view taken along the line C-C in FIG. 4.

As shown in FIGS. 2 and 6, the operation wire 22 and the power-supply wire 25 are disposed substantially parallel with each other inside the inner coil sheath 15 of the insertion part 12. In the embodiment, to reduce the outer diameter of the insertion part 12, the inner coil sheath 15 does not include partitions for partitioning the operation wire 22, the power-supply wire 25, and a storage space. Therefore, the outer peripheral surface of the operation wire 22 and the outer peripheral surface of the power-supply wire 25 can touch each other inside the inner coil sheath 15.

The distal end 22a of the operation wire 22 is fixed to the transmitting member 9 of the treatment part 2 (see FIG. 2). The proximal end 22b of the operation wire 22 is fixed to the slider 21 of the operation part 16 (see FIG. 1). The operation wire 22 includes a first core member 23 formed into a wire shape, and an insulating first covering member 24 that covers the outer peripheral surface of the first core member 23.

As the material for the first core member 23 it is possible to use a flexible material that has little elongation when pulled in the direction of its center axis, and high resistance to compression when pressed in the direction of its center axis. For example, a thin wire material or the like of metal or alloy can be used as the first core member 23.

The first covering member 24 is formed from an insulating material. For example, polyethylene (PE), PE elastomer, polyetheretherketone (PEEK), or fluorine resin can be used as the material for the first insulating member 24. As specific examples of fluorine resin, for example, polytetrafluoroethylene (PTFE), perfluoroalkoxy alkane (PFA), perfluoro ethylene-propylene copolymer (FEP), and the like, can be used.

As shown in FIGS. 1 and 4, the distal end 25a of the power-supply wire 25 is fixed to the first forceps member 3a of the treatment part 2. The proximal end 25b of the power-supply wire 25 is fixed to the terminal part 19 of the operation part 16.

As shown in FIGS. 5 and 6, the power-supply wire 25 includes a second core member 26 made from a conductor, and an insulating second covering member 27 that covers the outer peripheral surface of the second core member 26. In this embodiment, the insulating second covering member 27 insulates the power-supply wire 25 from the operation wire 22. Since the first covering member 24 also has an insulating effect, it further increases the insulation between the power-supply wire 25 and the operation wire 22.

As the material for the second core member 26, a material that allows a high-frequency current to be conducted can be used. For example, a thin wire material of metal or alloy can be used as the second core member 26. In the embodiment, the second core member 26 of the power-supply wire 25 is fixed to the treatment part 2 and the terminal part 19, and a high-frequency current can be supplied from the terminal part 19 and through the second core member 26 to the incision electrode 4 of the first forceps member 3a.

The second covering member 27 is made from a different type of material from that of the first covering member 24. The material for the second covering member 27 can be determined by selecting a different one from that used for the first covering member 24 from among polyethylene (PE), PE elastomer, polyetheretherketone (PEEK), and fluorine resin. That is, the combination of materials for the first covering member 24 and the second covering member 27 is selected such that, after the first core member 23 and the second core member 26 have been covered with the first covering member 24 and the second covering member 27 respectively, the surface conditions of the first covering member 24 and the second covering member 27 are mutually different.

Due to the mutually different surface conditions of the first covering member 24 and the second covering member 27, the coefficient of friction between the outer peripheral surface of the first covering member 24 and the outer peripheral surface of the second covering member 27 is different from the coefficient of friction between first covering members 24 and the coefficient of friction between second covering members 27.

In the embodiment, the distal end 25a of the power-supply wire 25 is connected via the first forceps member 3a and the covering member 7 to the distal end of the insertion part 12. The proximal end 25b of the power-supply wire 25 is fixed to the rotation-operation body 18 of the operation part 16. Consequently, while the operation wire 22 can move forward and backward within the insertion part 12, the power-supply wire 25 cannot do so. Since the slider 21 is moved forward and backward with respect to the rotation-operation body 18 in this manner, the operation wire 22 and the power-supply wire 25 move relatively.

Subsequently, the effects of the treatment instrument for endoscope 1 will be explained.

When using the treatment instrument for endoscope 1, the user moves the slider 21 of the operation part 16 shown in FIG. 1 forward and backward with respect to the rotation-operation body 18. An actuating force is thereby applied via the operation wire 22 to the pair of forceps members 3. When the operation wire 22 is moved to the distal-end side of the insertion part 12 while the pair of forceps members 3 are in the closed state, the pair of forceps members 3 will open (see FIG. 3). Conversely, when the operation wire 22 is moved to the proximal-end side of the insertion part 12 while the pair of forceps members 3 are in the open state, the pair of forceps members 3 will close.

The power-supply wire 25 fixed to the first forceps member 3a, which is the one of the pair of forceps members 3 where the incision electrode 4 is formed, does not move within the inner coil sheath 15 even if the pair of forceps members 3 open and close.

Therefore, if the operation wire 22 for opening and closing the pair of forceps members 3 is moved forward and backward within the inner coil sheath 15, the operation wire 22 and the power-supply wire 25 move relatively. At this time, the outer peripheral surface of the operation wire 22 and the outer peripheral surface of the power-supply wire 25 slide.

In this embodiment, the covering member of the operation wire 22 and the covering member of the power-supply wire 25 are made from different materials. The surface condition of the outer peripheral surface of the operation wire 22 and the surface condition of the outer peripheral surface of the power-supply wire 25 are mutually different due to the materials they are made from. That is, in this embodiment, the microscopic structure of the outer peripheral surface of the operation wire 22 and the microscopic structure of the outer peripheral surface of the power-supply wire 25 are mutually different.

For example, when the outer peripheral surface of the operation wire 22 and the outer peripheral surface of the power-supply wire 25 are made from the same material and formed in similar shapes, the microscopic structure of the outer peripheral surface of the operation wire 22 and the microscopic structure of the outer peripheral surface of the power-supply wire 25 will be substantially similar. In such a case, when the operation wire 22 and the power-supply wire 25 touch each other, there will be cases where the outer peripheral surface of the operation wire 22 and the outer peripheral surface of the power-supply wire 25 fit together or closely attach.

When the outer peripheral surface of the operation wire 22 and the outer peripheral surface of the power-supply wire 25 fit together or closely attach, the amount of actuating force required to open and close the pair of forceps members 3 by moving the operation wire 22 forward and backward is greater than when the operation wire 22 and the power-supply wire 25 are not touching each other. Specifically, the amount of actuating force increases by an amount equivalent to the frictional resistance between the covering member of the operation wire 22 and the covering member of the power-supply wire 25.

In contrast, in the embodiment, the outer peripheral surface of the operation wire 22 and the outer peripheral surface of the power-supply wire 25 have mutually different microscopic structures. Therefore, when the outer peripheral surfaces of the operation wire 22 and the power-supply wire 25 make contact with each other, a plurality of microscopic gaps are formed between them along their entire lengths. This configuration makes it more difficult for the outer peripheral surfaces of the operation wire 22 and the power-supply wire 25 to fit together or closely attach.

As a result, since the first covering member 24 of the operation wire 22 and the second covering member 27 of the power-supply wire 25 are made from mutually different materials, even if the operation wire 22 and the power-supply wire 25 touch each other, frictional resistance between the operation wire 22 and the power-supply wire 25 can be reduced. This makes it possible to move the operation wire 22 forward and backward with a smaller amount of actuating force than when the covering members of the operation wire 22 and the power-supply wire 25 are made from the same material.

Also, since the first covering member 24 and the second covering member 27 are made from different materials, there is less fictional force between their outer surfaces.

In this way, according to the treatment instrument for endoscope 1 of the embodiment, since the surface condition of the outer peripheral surface of the operation wire 22 and the surface condition of the outer peripheral surface of the power-supply wire 25 are different, sliding resistance between the operation wire 22 and the power-supply wire 25 can be reduced. This enables the treatment instrument for endoscope 1 to be operated even with a low amount of actuating force.

Since the operation wire 22 is formed by covering the first core member 23 with the first covering member 24, and the power-supply wire 25 is formed by covering the second core member 26 with the second covering member 27, there is high insulation between the operation wire 22 and the power-supply wire 25.

Due to the different microscopic structures of the outer peripheral surfaces of the first covering member 24 and the second covering member 27, their coefficients of friction are mutually different, as already mentioned. This can reduce the possibility that the outer peripheral surface of the first covering member 24 and the outer peripheral surface of the second covering member 27 will fit together or closely attach.

Furthermore, since the material of the first covering member 24 and the material of the second covering member 27 are mutually different, it is easy to configure covering members with mutually different surface conditions.

### Modified Examples

Subsequently, a first modified example of the treatment instrument for endoscope 1 of the embodiment will be explained.

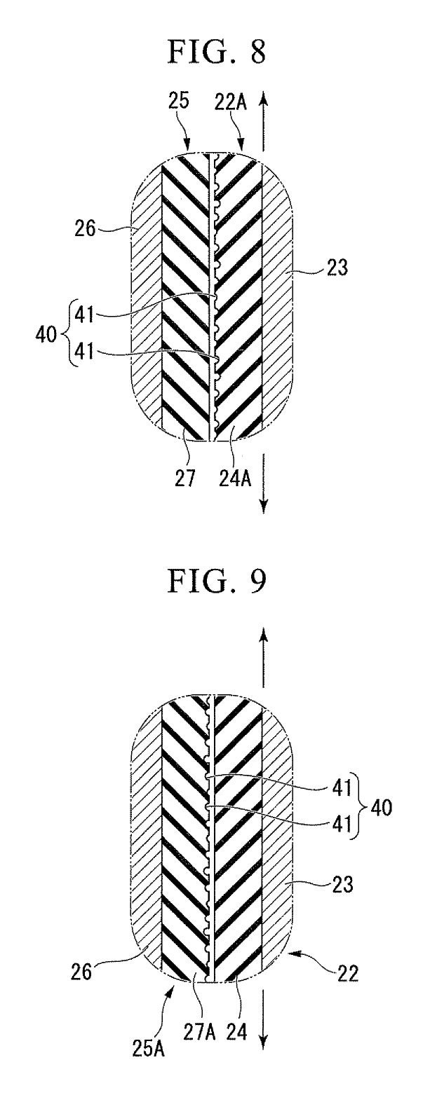

FIG. 7 is a view showing the configuration of one part of the treatment instrument for endoscope 1 of the modified example, and shows a cross-sectional view taken along the line D-D in FIG. 6. FIG. 8 is an enlarged view showing the section in FIG. 7 indicated by symbol X.

As shown in FIG. 7, a point of difference in the modified example is that, instead of the operation wire 22, an operation wire 22A is provided.

The operation wire 22A includes a first core member 23 and a first covering member 24A, The first covering member 24A differs from the first covering member 24 described above in that a first concavoconvex pattern 40 is formed in its outer peripheral surface. The first concavoconvex pattern 40 is formed by a method such as, for example, emboss processing during the step of covering the first core member 23 with the first covering member 24A. The first concavoconvex pattern 40 of this embodiment has a plurality of dimples 41 that are indented radially inward from the outer peripheral surface of the first covering member 24. The depth of the dimples 41 of the first concavoconvex pattern 40 is adjusted at the time of molding so that they do not form holes exposing the first core member 23 in the first covering member 24.

As shown in FIG. 8, in the modified example, the first concavoconvex pattern 40 is formed in the outer peripheral surface of the operation wire 22A. Thus, even if the outer peripheral surface of the operation wire 22A contacts the outer peripheral surface of the power-supply wire 25, at the dimples 41, gaps are formed between the outer peripheral surface of the operation wire 22A and the outer peripheral surface of the power-supply wire 25. The treatment part 2 can therefore be operated with a small amount of actuating force, as in the treatment instrument for endoscope 1 described above, without the outer peripheral surfaces of the operation wire 22A and the power-supply wire 25 closely attaching with each other.

In the modified example, since the dimples 41 forming the first concavoconvex pattern 40 change the frictional resistance between the outer peripheral surfaces of the first covering member 24A and the second covering member 27, the first covering member 24A and the second covering member 27 can be made from the same material.

FIG. 9 is a view of another configuration of the modified example described above (second modified example), and shows an enlarged view of a section corresponding to the section in FIG. 7 indicated by symbol X.

As shown in FIG. 9, when the operation wire 22 described in the embodiment is combined with a power-supply wire 25A including a second covering member 27A with the first concavoconvex pattern 40 of the modified example formed therein, similar effects to those of the modified example are obtained.

As yet another example configuration of the modified example described above (third modified example), two mutually different types of concavoconvex patterns (a first concavoconvex pattern and a second concavoconvex pattern) can be formed in the outer peripheral surfaces of the operation wire 22A and the power-supply wire 25.

For example, the first covering member 24 with a first concavoconvex pattern and the second covering member 27 with a second concavoconvex pattern can be formed on the operation wire 22 and the power-supply wire 25 using mutually different formation methods, so that the apertures of their dimples and the densities of their pluralities of dimples are different.

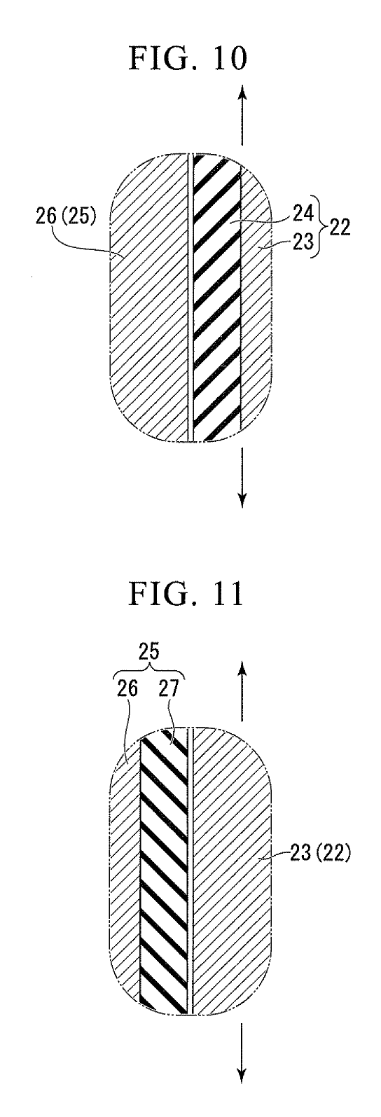

While an embodiment of the present invention has been described with reference to the drawings, the specific configuration is not limited to the embodiment, and includes design modifications and the like that do not depart from the main points of the invention. FIG. 10 is a view showing another example configuration (fourth modified example) of the treatment instrument for endoscope 1, and shows an enlarged view of a section corresponding to the section in FIG. 7 indicated by symbol X.

FIG. 11 is a view of yet another example configuration of a modified example (fifth modified example) of the treatment instrument for endoscope 1, and shows an enlarged view of a section corresponding to the section of FIG. 7 indicated by symbol X.

As shown in FIGS. 10 and 11, even if only one of the operation wire 22 and the power-supply wire 25 is covered, the surface conditions of the outer peripheral surface of the operation wire 22 and the outer peripheral surface of the power-supply wire 25 are different, and similar effects to those of the embodiment and modified examples described above are obtained.

While the embodiment described above is an example of a monopolar configuration including the operation wire 22 that current need not be passed through and the power-supply wire 25 that the high-frequency current is passed through, the treatment instrument for endoscope of this embodiment is not limited to a monopolar configuration. For example, the present invention can be favorably applied in a bipolar treatment instrument for endoscope, in which a pair of power-supply wires that high-frequency current is passed through are arranged within an insertion part, obtaining the effects of the invention described above.

While the embodiment describes an example where two wires are arranged inside a cylindrical member, three or more wires may be arranged inside one cylindrical member. In that case, the effects of the invention can be obtained by ensuring that those of the outer peripheral surfaces of the three or more wires that can contact each other have mutually different surface conditions.

The constituent elements of the embodiment and the modified examples described above can be combined as appropriate.

While preferred embodiments of the invention have been described above, the invention is not limited to these. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A treatment instrument for endoscope comprising:
a sheath that is capable of being inserted into a treatment instrument channel of an endoscope;
a treatment part provided on a distal-end side of the sheath;
a first wire that is connected to the treatment part and inserted into the sheath, the first wire comprising a first core member and a first covering member that covers the first core member; and
a second wire that drives the treatment part, connected to the treatment part and inserted into the sheath so that the second wire is capable of moving relatively with the first wire in a state where an outer peripheral surface of the second wire capable of contacting the first wire, the second wire comprising a second core member and a second covering member having a different surface condition from that of the first covering member.

2. The treatment instrument for endoscope according to claim 1, wherein
the first wire is a power-supply wire that supplies electrical power to the treatment part.

3. The treatment instrument for endoscope according to claim 1, wherein
outer peripheral surfaces of the first covering member and the second covering member have mutually different coefficients of friction.

4. The treatment instrument for endoscope according to claim 1, wherein
the first covering member and the second covering member are made from different materials.

5. The treatment instrument for endoscope according to claim 1, wherein
a first concavoconvex pattern is formed in the outer peripheral surface of the first covering member.

6. The treatment instrument for endoscope according to claim 5, wherein
a second concavoconvex pattern having a different shape to that of the first concavoconvex pattern is formed in the outer peripheral surface of the second covering member, by a formation method that is different from that of the first concavoconvex pattern.

7. The treatment instrument for endoscope according to claim 2, wherein
the first covering member is made from a material having an electrical insulating effect.

8. The treatment instrument for endoscope according to claim 1, wherein
the outer peripheral surfaces of the first covering member and the second covering member have mutually different microscopic structures.
